(19) United States
(12) Patent Application Publication
(10) Pub. No.: US 2017/0100046 A1
(43) Pub. Date: Apr. 13, 2017

(54) WEARABLE WIRELESS 12-CHANNEL ELECTROCARDIOGRAM SYSTEM

(71) Applicant: K-HEALTHWEAR CO., Ltd., Daejeon (KR)

(72) Inventors: Tae Hwan ROH, Suwon-si (KR); Ki Seok SONG, Seoul (KR); Hyun Woo CHO, Anyang-si (KR); Young Ho CHOI, Seoul (KR)

(73) Assignee: K-HEALTHWEAR CO., Ltd., Daejeon (KR)

(21) Appl. No.: 15/287,750

(22) Filed: Oct. 7, 2016

(30) Foreign Application Priority Data
Oct. 7, 2015 (KR) 10-2015-0141243

(51) Int. Cl. A61B 5/0245; A61B 5/0408; A61B 5/00

(52) U.S. Cl. CPC A61B 5/02455; A61B 5/0006; A61B 5/742; A61B 5/746; A61B 5/04085

(57) ABSTRACT

A wearable wireless 12-channel electrocardiogram system of the present invention includes: a wearable integrated electrocardiogram measurement device including a single electrode sheet having 10 electrodes and capable of being attached to a chest, and a micro-electrocardiogram measurement module detachably attached to and integrated with the electrode sheet, and configured to receive electrical signals from the 10 electrodes, to process the received signals and to transmit the processed signals to the outside; and a radio device including a controller configured to analyze and process electrocardiogram measurement information received from the wearable integrated electrocardiogram measurement device into 12 channels and to transmit the analyzed and processed electrocardiogram measurement information to an external server.

### TECHNICAL FIELD

The present invention relates to a wearable wireless 12-channel electrocardiogram system and, more specifically, to a wearable wireless 12-channel electrocardiogram system capable of continuously measuring a 12-channel electrocardiogram for a long time, storing and managing the measured 12-channel electrocardiogram by wearing an integrated electrode sheet, measuring the 12-channel electrocardiogram and wirelessly transmitting measured data to a radio device and an external server.

### BACKGROUND ART

With recent development of medical technology and an aging society, medical expenses are remarkably increasing. To reduce such medical expenses, conventional treatment-oriented medical technology gradually changes to diagnosis-and-prevention-oriented medical technology.

Particularly, cardiovascular disorders are chronic diseases that are difficult to fully recover from. Accordingly, it is very important to consistently measure and manage cardiovascular states to prevent and treat cardiovascular disorders.

Related Art 1 (Korean Patent 10-1002020) relates to a real-time electrocardiogram monitoring system and method, a patch type electrocardiograph and a communication device and discloses technology of continuously measuring a 1-channel electrocardiogram for a long time using a patch type electrocardiograph having three electrodes, and managing and storing the measured electrocardiogram.

Related Art 2 (Korean Patent 10-1381136) extends the number of electrocardiogram channels to 3 channels from the form of Related Art 1 (Korean Patent 10-1002020) and discloses technology of measuring, managing and storing an electrocardiogram of three channels larger than that in Related Art 1 (Korean Patent 10-1002020) for more accurate cardiovascular state diagnosis.

Related Art 3 (Korean Patent 10-1467351) relates to a 12-channel electrocardiogram electrode system and discloses technology with respect to wired electrodes attached over two sheets to measure a 12-channel electrocardiogram.

However, although related arts (Related Art 1 and Related Art 2) provide technology of conveniently measuring an electrocardiogram for a long time, accurate cardiovascular state diagnosis is impossible because the number of channels is limited to three.

In addition, while the related art (Related Art 3) provides technology for measuring up to a 12-channel electrocardiogram, it is difficult to conveniently measure, store and manage a 12-channel electrocardiogram for a long time because 12-channel electrodes divided into two sheets are connected to an external electrocardiogram device and 10 lines. Consequently, it is difficult to accurately diagnose a cardiovascular state.

Therefore, there is a need for development of a wearable electrocardiogram system capable of 1) constantly measuring an electrocardiogram for a long time and 2) measuring a multi-channel (e.g., 12-channel) electrocardiogram.

### RELATED ART REFERENCES

### Patent References

[Patent Reference 1] 1) Related Art 1 (Korean Patent 10-1002020)

[Patent Reference 2] 2) Related Art 2 (Korean Patent 10-1381136)

[Patent Reference 3] 1) Related Art 3 (Korean Patent 10-1467351)

### DISCLOSURE

### Technical Problem

An object of the present invention is to provide a wearable wireless 12-channel electrocardiogram system capable of continuously measuring a 12-channel electrocardiogram for a long time, storing and managing the measured electrocardiogram by wearing one integrated electrode sheet, measuring a 12-channel electrocardiogram and wirelessly transmitting measured data to an electrocardiogram controller and an external server.

### Technical Solution

To accomplish the object of the present invention, there is provided a wearable integrated electrocardiogram measurement device including: a single patch type electrode sheet having a plurality of electrodes formed on a sheet to be attached to a chest; and a micro-electrocardiogram measurement module directly attached to and integrated with the electrode sheet and configured to receive electrical signals from the electrodes, to process the received signals and to transmit the processed signals to the outside.

To accomplish the object of the present invention, there is also provided a wearable wireless 12-channel electrocardiogram system including: a wearable integrated electrocardiogram measurement device including a single electrode sheet having 10 electrodes and capable of being attached to a chest, and a micro-electrocardiogram measurement module detachably attached to and integrated with the electrode sheet, and configured to receive electrical signals from the 10 electrodes, to process the received signals and to transmit the processed signals to the outside; and a radio device including a controller configured to analyze and process electrocardiogram measurement information received from the wearable integrated electrocardiogram measurement device into 12 channels and to transmit the analyzed and processed electrocardiogram measurement information to an external server.

To accomplish the object of the present invention, there is also provided a wearable wireless 12-channel electrocardiogram system including: a wearable integrated electrocardiogram measurement device including a single electrode sheet having 10 electrodes and capable of being attached to a chest, and a micro-electrocardiogram measurement module detachably attached to and integrated with the electrode sheet, and configured to receive electrical signals from the 10 electrodes, to process the received signals and to transmit the processed signals to the outside; a radio device including a controller configured to analyze and process electrocardiogram measurement information received from the wearable integrated electrocardiogram measurement device into 12 channels and to transmit the analyzed and processed electrocardiogram measurement information to an external server; and a server configured to receive electrocardiogram signals and cardiovascular state data from the radio device, to store the electrocardiogram signals and cardiovascular state data and to transmit diagnosis results based on the electrocardiogram signals and cardiovascular state data to the radio device, wherein one of a default mode, a continuous reproduction mode and a device storage mode is realized depending on whether electrocardiogram data is transmitted in real time among the electrocardiogram measurement module, the radio device and the server.

### Advantageous Effects

The present invention has technical advantages of continuously measuring a 12-channel electrocardiogram, storing and managing measured electrocardiogram more accurately and conveniently.

In addition, the present invention has technical advantages of outputting a 12-channel electrocardiogram through an application for an electrocardiogram controller based on a smartphone carried by a user to allow the user to manage his or her cardiovascular state more conveniently.

Furthermore, the present invention has technical advantages of providing 12-channel electrocardiogram data of a user to external doctors and experts through a server, thereby achieving accurate cardiovascular state diagnosis.

### DESCRIPTION OF DRAWINGS

FIG. 1 illustrates a configuration of a wearable wireless 12-channel electrocardiogram system according to the present invention.

FIG. 2a illustrates an integrated electrode sheet for a 12-channel electrocardiogram according to a first embodiment of the present invention.

FIG. 2b illustrates an integrated electrode sheet for a 12-channel electrocardiogram according to a second embodiment of the present invention.

FIG. 2c illustrates an integrated electrode sheet for a 12-channel electrocardiogram according to a third embodiment of the present invention.

FIG. 3a is a front view of a mounted type electrocardiogram measurement module according to a first embodiment of the present invention.

FIG. 3b is a side view of the mounted type electrocardiogram measurement module according to the first embodiment of the present invention.

FIG. 3c is a front view illustrating an electrical connection state of pogo pins of a pogo pin contact part and electrodes according to the first embodiment of the present invention.

FIG. 4a is a front view of a clip type electrocardiogram measurement module according to a second embodiment of the present invention.

FIG. 4b is a side view of the clip type electrocardiogram measurement module according to the second embodiment of the present invention.

FIG. 4c is a rear view of the clip type electrocardiogram measurement module when a clip is closed according to the second embodiment of the present invention.

FIG. 4d is a rear view of the clip type electrocardiogram measurement module when the clip is open according to the second embodiment of the present invention.

FIG. 5a is a side view illustrating a coupling state of the mounted type electrocardiogram measurement module and the 12-channel electrocardiogram dedicated electrode sheet according to a first embodiment of the present invention.

FIG. 5b is a front view illustrating the coupling state of the mounted type electrocardiogram measurement module and the 12-channel electrocardiogram dedicated electrode sheet according to the first embodiment of the present invention.

FIG. 6a is a side view illustrating a coupling state of the clip type electrocardiogram measurement module and the 12-channel electrocardiogram dedicated electrode sheet according to a second embodiment of the present invention.

FIG. 6b is a front view illustrating the coupling state of the clip type electrocardiogram measurement module and the 12-channel electrocardiogram dedicated electrode sheet according to the second embodiment of the present invention.

FIG. 7 illustrates a configuration of the electrocardiogram measurement module according to the present invention.

FIG. 8 illustrates a configuration of a radio device according to an embodiment of the present invention.

FIG. 9a illustrates a state of a default mode from among operation modes of the wearable wireless 12-channel electrocardiogram system according to the present invention.

FIG. 9b illustrates a state of a continuous reproduction mode from among the operation modes of the wearable wireless 12-channel electrocardiogram system according to the present invention.

FIG. 9c illustrates a state of a device storage mode from among the operation modes of the wearable wireless 12-channel electrocardiogram system according to the present invention.

### BEST MODE

Detailed embodiments of the present invention will be described hereinbelow with reference to the attached drawings.

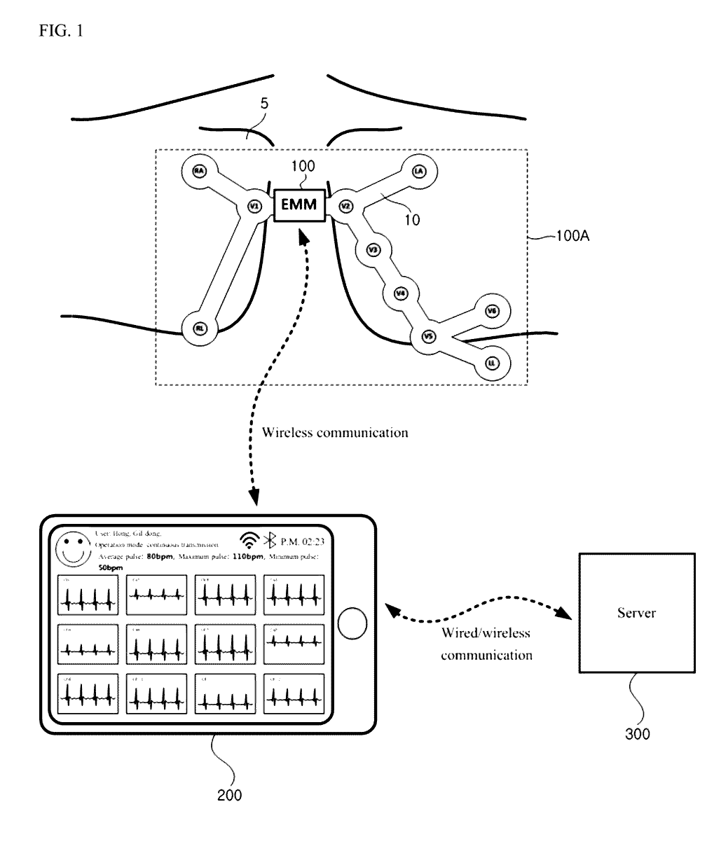

FIG. 1 illustrates a configuration of a wearable wireless 12-channel electrocardiogram system according to the present invention.

Referring to FIG. 1, the wearable wireless 12-channel electrocardiogram system according to the present invention includes an integrated electrocardiogram measurement device 100A, a radio device 200 and a server 300.

Here, the integrated electrocardiogram measurement device 100A includes a single patch type electrode sheet 10 having a plurality of electrodes formed on one sheet to be attached to the chest of a user, and a micro-electrocardiogram measurement module 100 that is directly attached to the electrode sheet to receive electrical signals from the electrodes, processes the electrical signals and then transmits the processed signals to the outside.

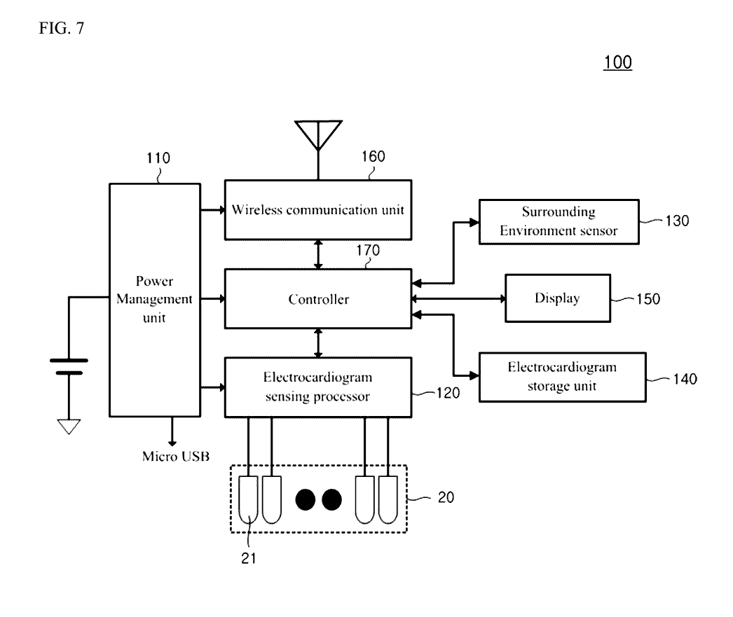

The electrocardiogram measurement module 100 is a micro device that executes functions of measuring electrocardiogram signals of a user or a patient, storing the measured signals and wirelessly transmitting the measured signals. The configuration and functions of the electrocardiogram device will be described in detail below with reference to FIG. 7.

In this case, the electrocardiogram measurement module 100 is attached to the chest 5 of a patient by being directly connected through pogo pins 21 of a pogo pin contact part 20 between first and second electrodes V1 and V2 from among 10 12-channel electrocardiogram dedicated electrodes attached to the chest 5 of the patient.

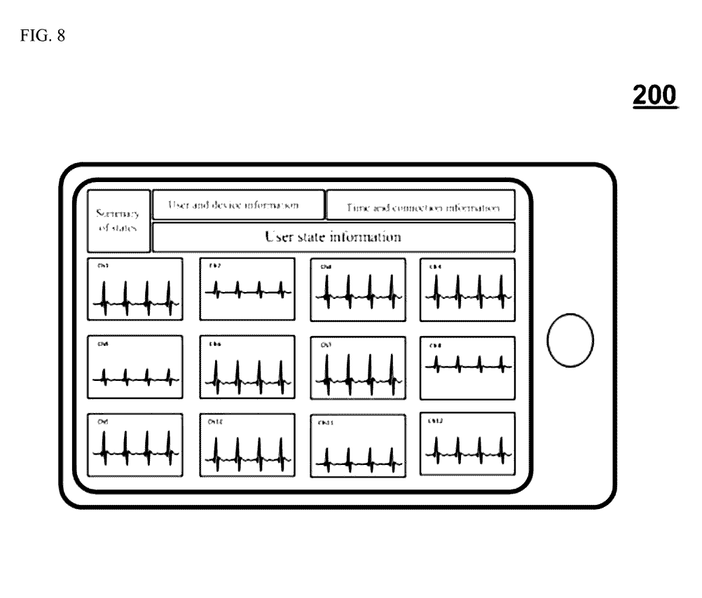

The radio device 200 processes electrocardiogram signals received from the electrocardiogram measurement module 100 and outputs 12-channel electrocardiogram signals and cardiovascular states (for example, an average heart rate, a maximum heart rate, a minimum heart rate, an instantaneous heart rate, etc.) in real time. A screen display method for the real-time output will be described in detail below with reference to FIG. 8.

The radio device 200 may control operation modes, gain and the like of the electrocardiogram measurement module 100 through wireless communication and transmits electrocardiogram signals and cardiovascular states of a user to the external server 300 of a hospital/specialized organization through wired/wireless communication.

In this case, Ethernet communication may be used as a wired communication method, and one or more of Wi-Fi, ZigBee, Bluetooth, RF, 3G, 4G, LTE, LTE-A and WiBro may be used as a wireless communication method. However, the present invention is not limited thereto.

The server 300 stores and manages electrocardiogram signals and cardiovascular states transmitted from the radio device 200. In addition, the server 300 can transmit a diagnosis result, obtained in such a manner that a doctor or an expert diagnoses a state of a patient on the basis of received electrocardiogram data, to the radio device 200 such that an alarm indicating an abnormal cardiovascular state of the patient is output.

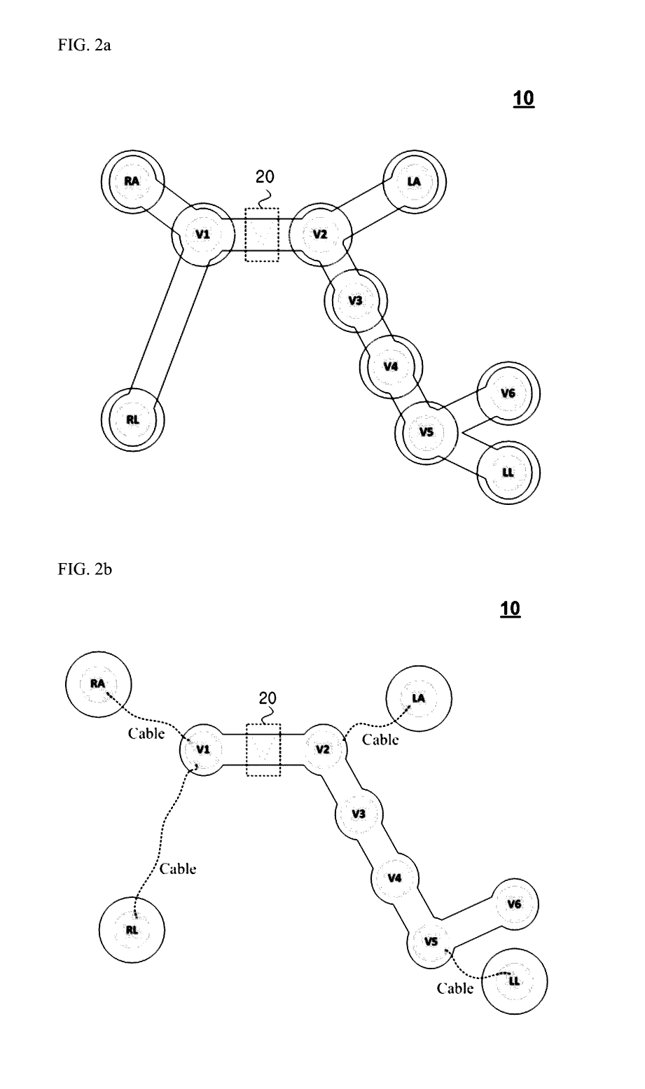

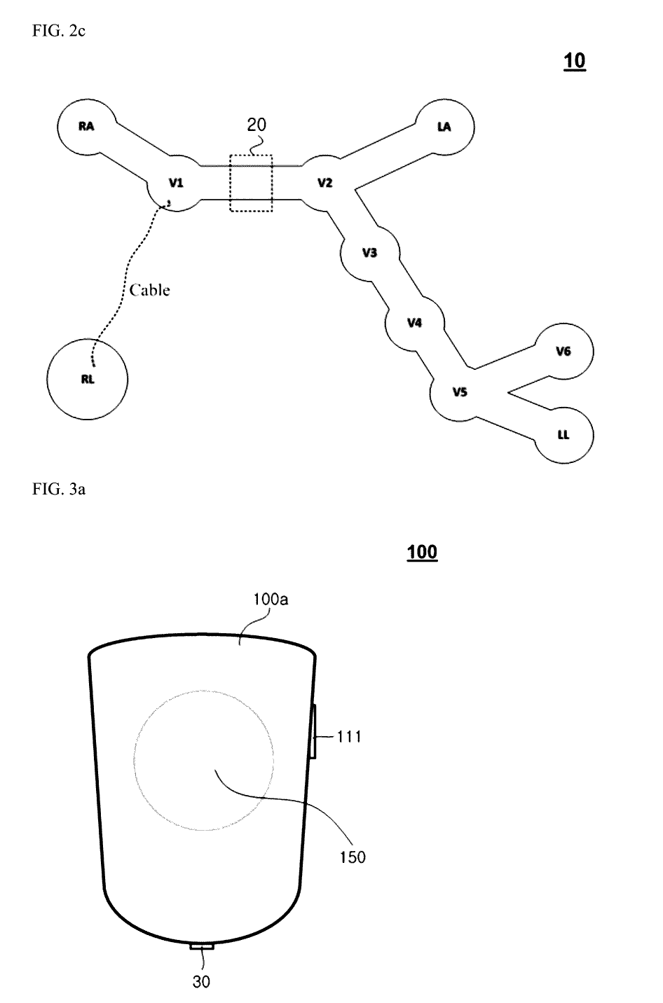

FIG. 2a illustrates an integrated electrode sheet for a 12-channel electrocardiogram according to a first embodiment of the present invention.

Referring to FIG. 2a, the integrated electrode sheet 10 for a 12-channel electrocardiogram according to the present invention includes 10 electrodes; first to sixth electrodes V1 to V6 and 4-limb electrodes RA, RL, LA and LL integrated on one sheet.

Connection relationships among the integrated electrode sheet 10 for a 12-channel electrocardiogram will now be described. The pogo pin contact part 20 is arranged between the first electrode V1 and the second electrode V2, the first electrode V1 is electrically connected to the first 4-limb electrode RA and the second 4-limb electrode RL having predetermined distances therefrom, and the second electrode V2 is electrically connected to the third 4-limb electrode LA and the third electrode V3 having predetermined distances therefrom. In addition, the third electrode V3 is electrically connected to the fourth electrode V4 having a predetermined distance therefrom, the fourth electrode V4 is electrically connected to the fifth electrode V5 having a predetermined distance therefrom, and the fifth electrode V5 is electrically connected to the fourth 4-limb electrode LL and the sixth electrode V6 having predetermined distances therefrom.

For 12-channel electrocardiogram measurement, 10 electrodes are required. Here, arrangement positions of the 10 electrodes are not fixed, and electrode positions may be determined in terms of body structure, as shown in Table 1.

Table 1 shows examples of portions of the body of a patient to which the 10 electrodes of the integrated electrode sheet 10 for 12-channel electrocardiogram are attached.

TABLE 1

| Electrode | Electrode position |
|---|---|
| V1 | Next to right sternum in the fourth intercostal space (between |
| | fourth and fifth ribs) |
| V2 | Next to left sternum in the fourth intercostal space (between |
| | fourth and fifth ribs) |
| V3 | Between V2 and V4 |
| V4 | Midclavicular line in the fifth intercostal space (between the |
| | fifth and sixth ribs) |
| V5 | Parallel with V4 and on the left armpit |
| V6 | Parallel with V4 and V5 and on the middle armpit line |
| RA | On the right arm, avoiding thick muscle |
| LA | On the left arm at the same position as that on the right arm |
| RL | On the right leg and next to calf muscles |
| LL | On the left leg at the same position as that on the right leg |

Accordingly, there is no standard for absolute positions of the 12-channel electrocardiogram electrodes, commonly used for all users to be measured, and the 10 electrodes may be arranged at different positions depending on body shapes of users to be measured.

However, to minimize the electrode size for convenience of users to be measured, it is desirable to respectively locate the 4-limb electrodes RA, RL, LA and LL at upper right, lower right, upper left and lower left of a square formed by the first to fourth electrodes V1 to V4 according to consultation of cardiology, for example.

That is, since the 4-limb electrodes RA, RL, LA and LL are generally located closer to the electrodes V1 to V6 than in 12-channel electrocardiogram electrode arrangement used in hospitals, it is necessary to locate the 4-limb electrodes RA, RL, LA and LL on the right arm, left arm, right leg and left leg instead of the chest, as shown in Table 1, for more accurate 12-channel electrocardiogram measurement.

The principle of measurement of 12-channel electrocardiogram using the 10 electrodes will be briefly described.

The following table 2 shows measurement information of 12 channels when a 12-channel electrocardiogram is measured using the 10 electrodes.

TABLE 2

| | |
|---|---|
| Channel I | LA − RA |
| Channel II | LL − RA |
| Channel III | LL − RA − LA = channel II − channel I |
| V1 | V1 − WCT (average of RA, LA and LL) |
| V2 | V2 − WCT (average of RA, LA and LL) |
| V3 | V3 − WCT (average of RA, LA and LL) |
| V4 | V4 − WCT (average of RA, LA and LL) |
| V5 | V5 − WCT (average of RA, LA and LL) |
| V6 | V6 − WCT (average of RA, LA and LL) |
| aVR | RA − (LA + LL)/2 |
| aVL | LA − (RA + LL)/2 |
| aVF | LL − (RA + LA)/2 |

Referring to Table 2, when the 10 electrodes V1 to V6, RA, RL, LA and LL transfer voltages measured therein to the micro-electrocardiogram measurement module 100 through pogo pins, the electrocardiogram measurement module 100 obtains 12-channel information by combining the received voltages of the respective electrodes and transmits the obtained 12-channel electrocardiogram information to the external radio device 200.

Specifically, the electrocardiogram measurement module 100 transmits the following 8-channel electrocardiogram compressed from the 12-channel electrocardiogram to the external radio device 200.

Lead V1=V1−WCT \textcircled{1}

Lead V2=V2−WCT \textcircled{2}

Lead V3=V3−WCT \textcircled{3}

Lead V4=V4−WCT \textcircled{4}

Lead V5=V5−WCT \textcircled{5}

Lead V6=V6−WCT \textcircled{6}

Lead I=LA−RA \textcircled{7}

Lead II=LL−RA \textcircled{8}

Then, the electrocardiogram measurement module 100 additionally generates a 4-channel electrocardiogram from the 8-channel electrocardiogram through the following expressions to accomplish 12-channel electrocardiogram information and transmits the 12-channel electrocardiogram information to the external radio device 200.

Lead III=Lead II−Lead I \textcircled{1}

aVR=(Lead I+Lead II)/2 \textcircled{2}

aVL=Lead I−Lead II/2 \textcircled{3}

aVF=Lead II−Lead I/2 \textcircled{4}

The integrated 12-channel electrocardiogram dedicated electrode sheet 10 is electrically connected to the micro-electrocardiogram measurement module 100 through the pogo pin contact part 20 which is electrically connected to the pogo pins 21 and located at the center between the first electrode V1 and the second electrode V2 corresponding to the solar plexus from among the ten electrodes.

The pogo pin contact part 20 may include 10 pogo pins for electrical connection with the 10 electrodes and may be arranged in arbitrary forms including a V shape and a parallel-line shape. An adhesive is coated on the bottom surfaces of the electrodes except connecting lines for connecting the electrodes of the integrated 12-channel electrocardiogram electrode sheet 10 such that the electrodes can be easily attached to the chest of a patient.

In this case, the adhesive may be coated only on portions around some electrodes or on all electrodes.

FIG. 2b illustrates an integrated electrode sheet for a 12-channel electrocardiogram according to a second embodiment of the present invention.

Referring to FIG. 2b, the integrated electrode sheet 10 for a 12-channel electrocardiogram according to the present invention includes 10 electrodes, that is, first to sixth electrodes V1 to V6 and 4-limb electrodes RA, RL, LA and LL, which are integrated on one sheet. Particularly, the 4-limb electrodes RA, RL, LA and LL are connected through cables.

The 4-limb electrodes RA, RL, LA and LL are respectively disposed at top/bottom and left/right of a square formed by the first to fourth electrodes V1 to V4 arranged to correspond to the heart portion of the user, in general. As illustrated in FIG. 2b, the first 4-limb electrode RA is connected to the first electrode V1 through a cable at the upper left side, the second 4-limb electrode RL is connected to the first electrode V1 through a cable at the lower left side, the third 4-limb electrode LA is connected to the second electrode V2 through a cable at the upper right side, and the fourth 4-limb electrode LL is connected to the fifth electrode V5 through a cable at the lower right side.

Although there are various standards for positioning the 4-limb electrodes RA, RL, LA and LL, patients have different body structures, sexes, ages and so on, and thus it is desirable to connect the 4-limb electrodes through the sheet, cables and the like without fixing the positions thereof.

Particularly, to accurately measure a 12-channel electrocardiogram for a user who hardly moves, such as a hospital inpatient, it is necessary to arrange the 4-limb electrodes RA, RL, LA and LL as widely as possible. In this case, cables integrated with the sheet are used, and thus the user need not connect additional lines.

FIG. 2c illustrates an integrated electrode sheet for a 12-channel electrocardiogram according to a third embodiment of the present invention.

Referring to FIG. 2c, the integrated electrode sheet 10 for a 12-channel electrocardiogram according to the present invention includes 10 electrodes, that is, first to sixth electrodes V1 to V6 and 4-limb electrodes RA, RL, LA and LL, which are integrated on one sheet. Particularly, only the second 4-limb electrode RL that is the farthest from the first to sixth electrodes V1 to V6 is connected to the first electrode V1 through a line such as a cable.

Characteristics of the integrated electrode sheet 10 for a 12-channel electrocardiogram according to the present invention are summarized through the embodiments illustrated in FIGS. 2a, 2b and 2c.

Firstly, the integrated electrode sheet 10 for a 12-channel electrocardiogram according to the present invention has 10 electrodes V1 to V6, RA, RL, LA and LL which are integrated without connection using additional lines and is worn by a patient to provide a technique of measuring a 12-channel electrocardiogram of the patient.

Accordingly, the present invention does not require additional connection of a measured user with the electrocardiogram measurement module 100 through a line, and thus the user can conveniently measure an electrocardiogram and noise generation is reduced, achieving more accurate 12-channel electrocardiogram measurement.

In addition, the present invention attaches electrodes to a fixed portion of a patient only once using the integrated electrodes and measures a 12-channel electrocardiogram while the patient wears the measurement device, thereby measuring electrocardiograms with higher detection sensitivity.

Secondly, the integrated electrode sheet 10 for a 12-channel electrocardiogram according to the present invention achieves electrical connection with the electrocardiogram device 100 through the pogo pin contact part 20.

Thirdly, under the condition that the integrated electrode sheet 10 for a 12-channel electrocardiogram according to the present invention has the integrated electrodes and the pogo pin contact part 20, electrode arrangement, line connection and the like can be freely determined.

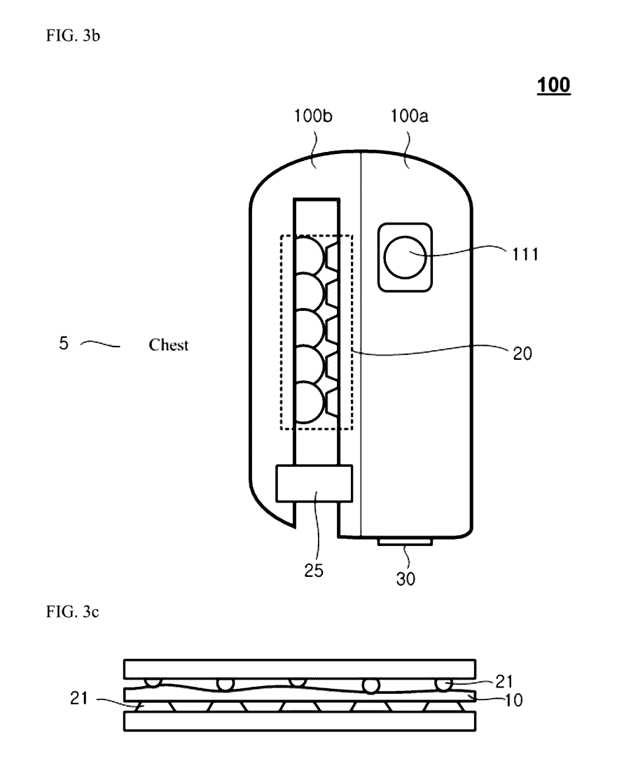

FIGS. 3a and 3b are, respectively, a front view and a side view of a mounted type electrocardiogram measurement module according to a first embodiment of the present invention.

Referring to FIGS. 3a and 3b, the mounted type electrocardiogram measurement module 100 according to the present invention includes a power switch 111, a display 150, the pogo pin contact part 20, a fixing part 25 and a USB port 30.

The power switch 111 is provided to the side of the front part 100a of the electrocardiogram measurement module and is used for power control. For example, a push switch, a slide switch or the like may be used as the power switch 11. However, the present invention is not limited thereto.

The display 150 is arranged at the center of the front part 100a of the electrocardiogram measurement module and displays power and battery states, electrodes and device attachment states, a cardiovascular state of a measured user, a system operation mode, a Bluetooth connection state and so on, for example, using colors and flickering speed of LED array. For example, an LCD, LED or the like may be used as the display 150. However, the present invention is not limited thereto.

The pogo pin contact part 20 is arranged at one side of the rear side 100b of the mounted type electrocardiogram measurement module and connects the mounted type electrocardiogram measurement module 100 to the integrated 12-channel electrocardiogram dedicated electrode sheets 10 through the pogo pins 21, as illustrated in FIG. 3c.

The fixing part 25 applies pressure such that the electrodes are fixed after contacting the pogo pins. As a method of applying pressure for stable operation, for example, a method of applying pressure using magnetic force by arranging magnets, a method of using a small clip, a method of connecting a band to the device or the like may be used. However, the present invention is not limited thereto.

FIG. 3c is a front view illustrating an electrical connection state of the pogo pins of the pogo pin contact part and the electrodes according to the first embodiment of the present invention.

Referring to FIG. 3c, the pogo pins 21 serve as contact pins that allow movement within a specific range when a predetermined degree of pressure is applied thereto. According to this function of the pogo pins 21, even when a gap is generated between the electrodes of the electrode sheet 10 inserted into the micro-electrocardiogram measurement module 100 and the pogo pins 21, the electrodes can contact the pogo pins 21 if the gap is within the movement range of the pogo pins 21 and thus the electrodes and the pogo pins 21 can be electrically connected to each other.

The USB port 30 is provided to the lower end of the front part 100a of the electrocardiogram measurement module and performs data transmission and reception by being connected with an external universal serial bus (USB). An internal battery of the electrocardiogram measurement module is charged through the USB port.

Additionally, an electrocardiogram measurement module circuit board (not shown) and the battery (not shown) are included inside of the front part 100a of the mounted type electrocardiogram measurement module 100, and the circuit board (not shown) of the front part 100a is electrically connected to the pogo pin contact part 20 of the rear side 100b using an F-PCB, a PCB, a conductive material or the like.

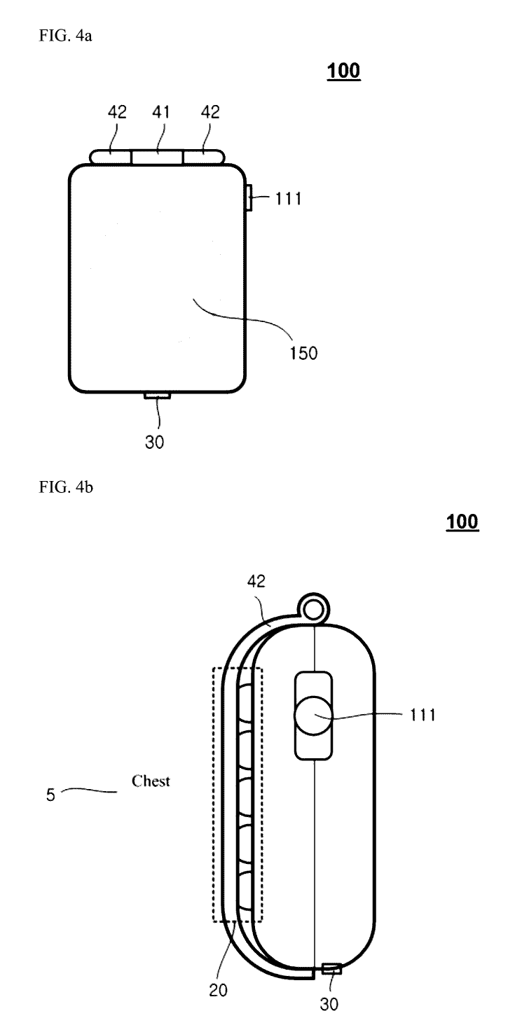

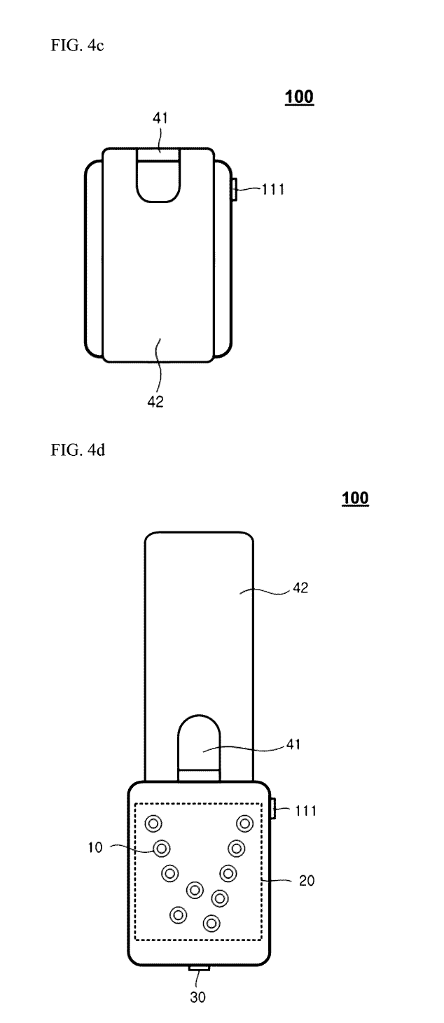

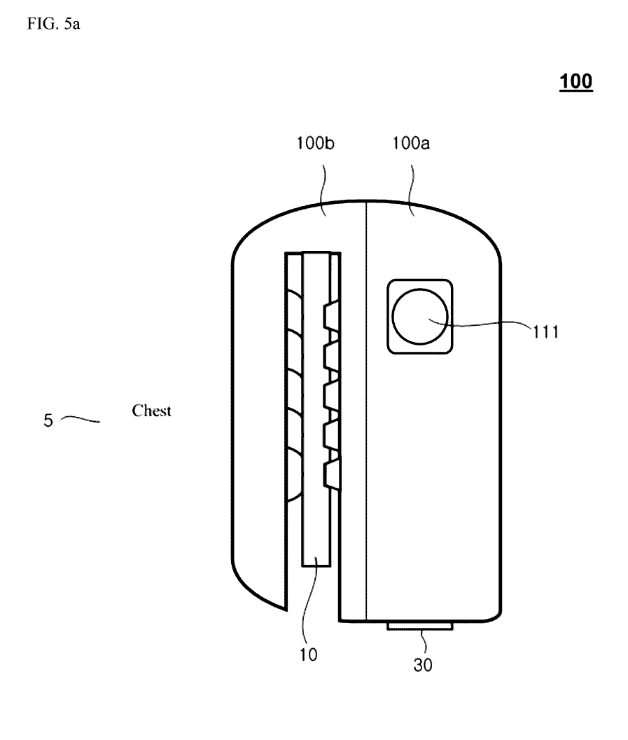

FIGS. 4a and 4b are, respectively, a front view and a side view of a clip type electrocardiogram measurement module according to a second embodiment of the present invention.

FIG. 4c is a rear view of the clip type electrocardiogram measurement module when a clip is closed and FIG. 4d is a rear view of the clip type electrocardiogram measurement module when the clip is open.

Referring to FIGS. 4a to 4d, the clip type electrocardiogram measurement module 100 according to the present invention includes a clip rotating part 41, a fixing clip 42, the power switch 111, the display 150, the pogo pin contact part 20 and the USB port 30.

The clip rotating part 41 is provided to the upper end of the clip type electrocardiogram measurement module 100 and rotates the fixing clip 42 disposed in proximity thereto.

The fixing clip 42 is a rectangular plate having portions respectively disposed on both sides of the clip rotating part 41 and fixes the integrated 12-channel electrocardiogram dedicated electrode sheet 10 and the clip type electrocardiogram measurement module 100.

Accordingly, the clip type electrocardiogram measurement module 100 according to the present invention rotates the fixing clip 42 through the clip rotating part 41 to attach/detach the electrode sheet to/from the pogo pin contact part 20 (refer to FIGS. 4c and 4d).

The power switch 111, the display 150, the pogo pin contact part 20 and the USB port 30 have been described with reference to FIGS. 3a, 3b and 3c and description thereof is thus omitted.

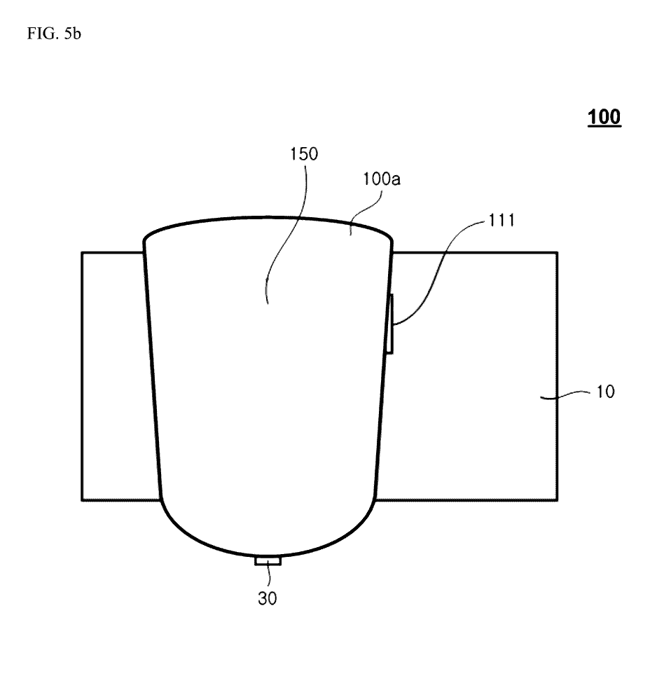

FIGS. 5a and 5b are respectively a side view and a front view illustrating a coupling state of the mounted type electrocardiogram measurement module and the 12-channel electrocardiogram dedicated electrode sheet according to a first embodiment of the present invention.

Referring to FIGS. 5a and 5b, the mounted type electrocardiogram measurement module 100 is coupled with the integrated 12-channel electrocardiogram dedicated electrode sheet 10 in such a manner that the mounted type electrocardiogram measurement module 100 is hung on the integrated 12-channel electrocardiogram dedicated electrode sheet 10.

In this case, the respective electrodes can be easily attached to the chest 5 of a patient because the predetermined adhesive material is coated on the lower surfaces of some or all electrodes except lines connecting electrodes of the integrated 12-channel electrocardiogram dedicated electrode sheet 10.

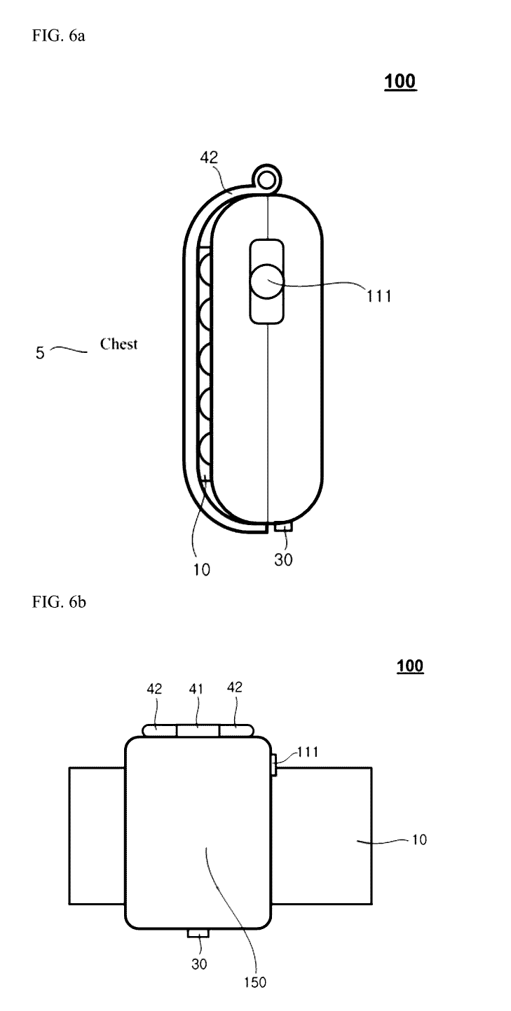

FIGS. 6a and 6b are, respectively, a side view and a front view illustrating a coupling state of the clip type electrocardiogram measurement module and the 12-channel electrocardiogram dedicated electrode sheet according to a second embodiment of the present invention.

Referring to FIGS. 6a and 6b, the clip type electrocardiogram measurement module 100 is coupled with the integrated 12-channel electrocardiogram dedicated electrode sheet 10 in such a manner that the integrated 12-channel electrocardiogram dedicated electrode sheet 10 is fixed by the fixing clip 42.

In this case, the respective electrodes can be easily attached to the chest 5 of a patient because the predetermined adhesive material is coated on the lower surfaces of some or all electrodes except lines connecting electrodes of the integrated 12-channel electrocardiogram dedicated electrode sheet 10.

FIG. 7 illustrates a configuration of the electrocardiogram measurement module according to the present invention.

Referring to FIG. 7, the electrocardiogram measurement module according to the present invention includes a power management unit 110, an electrocardiogram sensing processor 120, a surrounding environment sensor 130, an electrocardiogram storage unit 140, the display 150, a wireless communication unit 160 and a controller 170.

The power management unit 110 receives power from the battery of the electrocardiogram measurement module, provides power to each component and manages battery charging.

The electrocardiogram sensing processor 120 amplifies an electrocardiogram signal of a patient, input from the integrated 12-channel electrocardiogram dedicated electrode sheet 10 through the pogo pins 21 of the pogo pin contact part 20, filters noise from the electrocardiogram signal and converts the analog signal, that is, the filtered electrocardiogram signal, into a digital signal.

The surrounding environment sensor 130 senses the temperature and humidity of a patient's room, the body temperature of the patient, and so on.

The electrocardiogram storage unit 140 stores electrocardiogram data of the patient, sensed and signal-processed in the electrocardiogram sensing processor 120. As a memory for storage, for example, a secure digital (SD) card, a micro SD card, a flash memory or the like may be used. However, the present invention is not limited thereto.

The display 150 is arranged at the center of the front part 100a of the electrocardiogram measurement module and displays power and battery states, electrode and device attachment states, cardiovascular state of a measured user, a system operation mode, a Bluetooth connection state and so on, for example, using colors and flickering speed of an LED array. For example, an LCD, LED or the like may be used as the display 150. However, the present invention is not limited thereto.

The wireless communication unit 160 provides an interface for transmitting electrocardiogram data measured by the measurement module 100 to the radio device 200 and receiving a control signal from the radio device 200. For example, ZigBee, RF, Wi-Fi, 3G, 4G, LTE, LTE-A, WiBro or the like may be used as a wireless communication scheme. However, the present invention is not limited thereto.

The controller 170 controls the power management unit 110, the electrocardiogram sensing processor 120, the surrounding environment sensor 130, the electrocardiogram storage unit 140, the display 150 and the wireless communication unit 160.

FIG. 8 illustrates a configuration of the radio device according to an embodiment of the present invention.

A screen composition and operation of the radio device according to the present invention will be described in detail with reference to FIGS. 1 and 8.

FIG. 8 illustrates a screen composition of an electrocardiogram controller dedicated application based on a popular radio device such as a smartphone, a tablet or a PC. In this case, the dedicated application is executed after user login and refers to user information through wired/wireless communication with the external server 300.

The electrocardiogram controller dedicated application of the radio device outputs 12-channel (ch1 to ch12) electrocardiogram signals in real time by default, outputs cardiovascular state information (e.g., average heart rate, maximum heart rate, minimum heart rate, stress index, etc.) of a current user, and outputs summary information about electrocardiogram states (e.g., normal/abnormal) through a display method such as a method of using a smile indication (refer to FIG. 1) or red/green signal light.

As the user and device information item, the name, sex and age of a user and an electrocardiogram measurement module operation mode (e.g., default mode, continuous reproduction mode or a device storage mode) are displayed.

As the time and connection information item, a wired/wireless network connection state between the radio device 200 and the electrocardiogram measurement module 100 or between the radio device 200 and the external server 300 is displayed.

Referring to FIG. 1, the radio device 200 may receive a diagnosis result transmitted from the external server 300 through a wired/wireless network and output an alarm. In addition, when no electrocardiogram signal is output from some of the 12 channels, the radio device 200 may sense that no electrocardiogram signal is output and output an alarm indicating that electrode attachment state is abnormal.

In the case of an abnormal cardiovascular state, the radio device 200 outputs an abnormal cardiovascular state alarm signal and transmits the abnormal cardiovascular state alarm signal to the external server 300.

Here, the abnormal cardiovascular state refers to a case in which the current heart rate or a heart rate variation of a user deviates from a normal heart rate or heart rate variation set by a doctor or an expert related to the external server 300.

As a method of outputting the alarm, for example, visual alarm output through a display, acoustic alarm output as sound, tactile alarm through vibration or the like may be used. However, the present invention is not limited thereto.

The main functions of the radio device including the electrocardiogram controller dedicated application according to the present invention will be summarized.

Firstly, the radio device according to the present invention executes the 12-channel electrocardiogram transmission/reception function, that is, the function of transmitting a 12-channel electrocardiogram received from the electrocardiogram measurement module 100 to the external server 300 through a wired/wireless network.

Secondly, the radio device according to the present invention executes the function of controlling the 12-channel electrocardiogram measurement module, that is, the function of setting an operation mode of the 12-channel electrocardiogram measurement module and controlling a gain thereof.

Thirdly, the radio device according to the present invention executes the function of analyzing 12-channel electrocardiograms, that is, the function of analyzing 12-channel electrocardiograms transmitted from the electrocardiogram measurement module 100 to extract a cardiovascular state index (e.g., current heart rate, average heart rate, maximum heart rate, minimum heart rate, instantaneous heart rate or the like).

In this case, the radio device according to the present invention may additionally extract a ventricular ectopic beat (VEB), ventricular flutter or fibrillation (VF), supraventricular ectopic beats (SVEB), atrial flutter or fibrillation (AF) or the like using an automatic analysis function.

Fourthly, the radio device according to the present invention executes the cardiovascular state output function, such as a function of outputting a 12-channel electrocardiogram analysis result and a cardiovascular state received from a doctor or an expert related to the external server 300 to a relevant measured user.

Fifthly, the radio device according to the present invention executes the function of announcing an electrode state and a device attachment state, for example, the function of outputting an alarm to the measured user when an electrode state and device attachment state are abnormal.

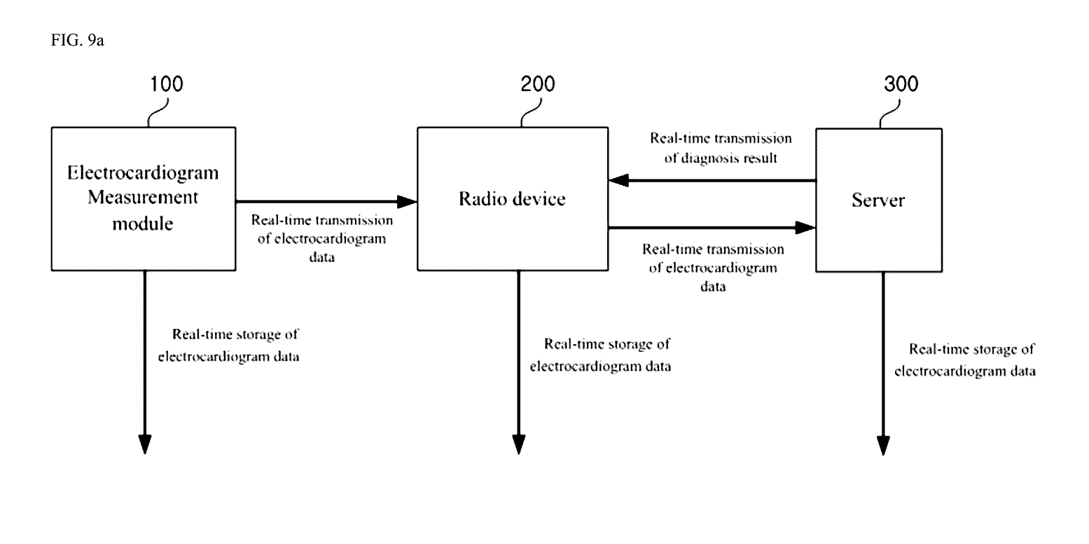

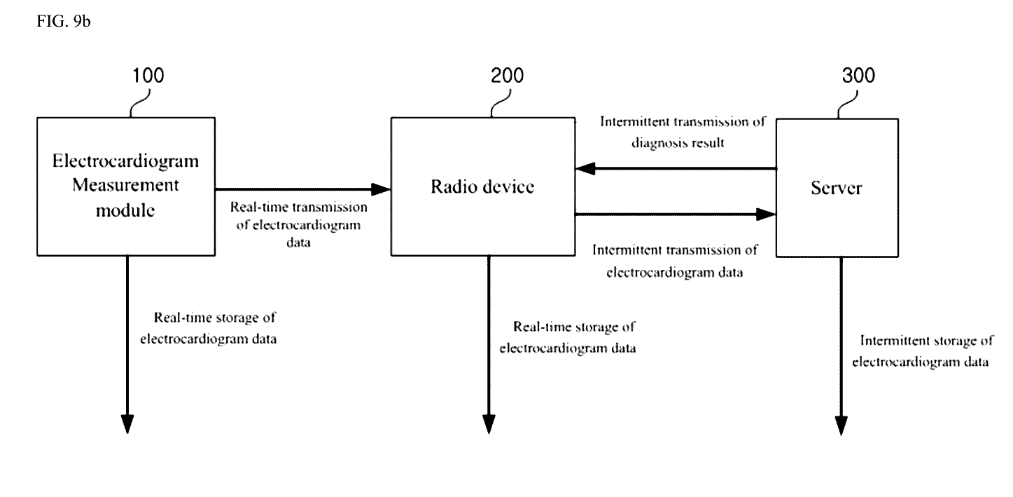

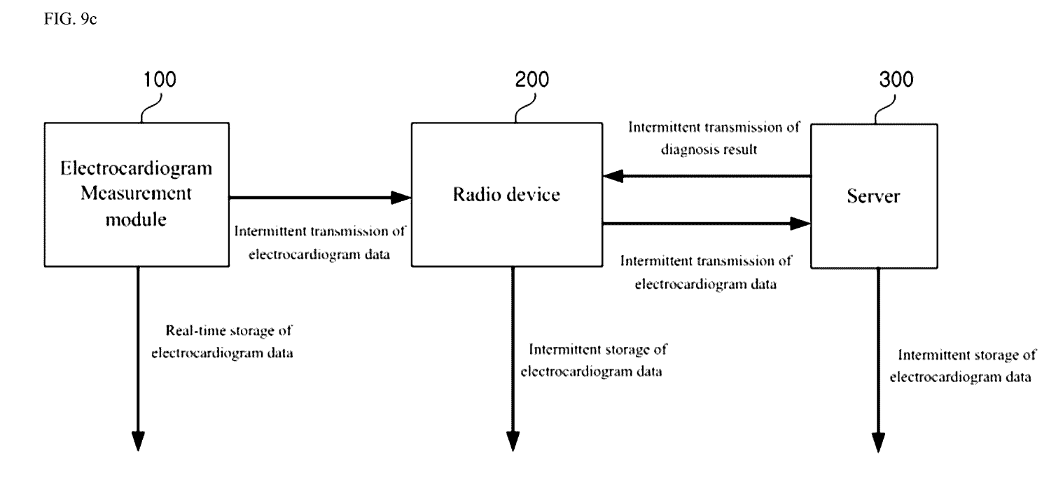

FIG. 9a illustrates a state of a default mode from among operation modes of the wearable wireless 12-channel electrocardiogram system according to the present invention.

Referring to FIG. 9a, the default mode of the wearable wireless 12-channel electrocardiogram system according to the present invention is a mode in which electrocardiogram data measured by the electrocardiogram measurement module 100 is transmitted to the radio device 200 in real time, delivered from the radio device 200 to the external server 300 in real time and stored in the electrocardiogram measurement module 100, the radio device 200 and the external server 300 in real time.

Here, the electrocardiogram data measured by the electrocardiogram measurement module 100 is transmitted to the external server 300 through the radio device 200 in order to decrease communication load (e.g., communication distance, the quantity of data or the like) of the electrocardiogram measurement module 100 to enable extended operation at low power even using a small-capacity battery.

In this case, the external server 300 transmits a diagnosis result to the radio device 200 when an abnormal signal is generated on the basis of the electrocardiogram data transmitted and stored in real time.

Specifically, the external server 300 stores electrocardiogram information of cardiovascular patients and normal persons as well as 12-channel electrocardiogram information of a large number of measured persons, distinguished from the electrocardiogram measurement module 100 that stores only 12-channel electrocardiogram information of one measured person.

That is, the external server 300 can perform additional analysis based on big data, which cannot be performed by the radio device 200 only. For example, since an electrocardiogram having a specific pattern is generated mostly due to arrhythmia, the external server 300 can provide an alarm for recommending additional arrhythmia examination to relevant patients.

Furthermore, when analysis requests for results that are difficult to judge through analysis of the radio device 200 or the automatic analysis function are received through the external server 300 installed in a hospital or a specialized organization, doctors or experts can directly process the analysis requests and feed back results to measured persons.

The default mode is an operation mode particularly suitable for patients in hospitals for whom all electrocardiogram data needs to be continuously measured, stored and managed in real time.

FIG. 9b illustrates a state of a continuous reproduction mode from among the operation modes of the wearable wireless 12-channel electrocardiogram system according to the present invention.

Referring to FIG. 9b, the continuous reproduction mode of the wearable wireless 12-channel electrocardiogram system according to the present invention is an operation mode in which electrocardiogram data measured by the electrocardiogram measurement module 100 is transmitted to the radio device 200 in real time and stored in the radio device 200 in real time.

That is, in the continuous reproduction mode, the electrocardiogram measurement module 100 and the radio device 200 store all electrocardiogram data in real time and the radio device 200 intermittently transmits electrocardiogram data to the external server 300.

The external server 300 intermittently receives electrocardiogram data from the radio device 200, intermittently stores the electrocardiogram data and intermittently sends diagnosis results to the radio device 200.

Here, “intermittent” refers to a case in which, although electrocardiogram data is stored in the electrocardiogram measurement module 100 and the radio device 200 in real time, some or all electrocardiogram data or diagnosis results are periodically or aperiodically transmitted between the radio device 200 and the external server 300 or transmission is performed when electrocardiogram data is abnormal.

For example, a case in which part of data is periodically transmitted may correspond to a case in which only electrocardiogram data corresponding to a section deviated from a specific heart rate range is transmitted at hourly intervals, and a case in which all data is aperiodically transmitted may correspond to a case in which all measured data is transmitted from the radio device 200 to the external server 300 at a time desired by a user.

More specifically, a case in which electrocardiogram data is measured for a long time during daily life and then stored data is transmitted from the radio device 200 to the external server 300 at once may be exemplified.

In the case of aperiodic transmission, transmission timing may be determined by a user, determined by the radio device 200 upon generation of an abnormal signal or determined at the request of a doctor or an expert in charge of the external server 300.

FIG. 9c illustrates a state of a device storage mode from among the operation modes of the wearable wireless 12-channel electrocardiogram system according to the present invention.

Referring to FIG. 9c, the device storage mode of the wearable wireless 12-channel electrocardiogram system according to the present invention is a mode in which all electrocardiogram data is stored in the electrocardiogram measurement module 100 in real time and the electrocardiogram measurement module 100 intermittently transmits electrocardiogram data to the radio device 200 and the external server 300.

Specifically, in the device storage mode, the radio device 200 intermittently receives electrocardiogram data from the electrocardiogram measurement module 100, intermittently stores electrocardiogram data and intermittently sends electrocardiogram data to the external server 300.

The external server 300 intermittently receives electrocardiogram data from the radio device 200, intermittently stores the electrocardiogram data and intermittently sends diagnosis results to the radio device 200.

Here, “intermittent” refers to a case in which a part of or all data is transmitted periodically or aperiodically like in the continuous reproduction mode, and a detailed description thereof is omitted.

The device storage mode is suitable for users in environments in which it is difficult to charge a battery for a long time in daily life because the wireless communication unit of the electrocardiogram measurement module 100 is intermittently used and thus power consumption can be reduced.

While the electrocardiogram measurement module 100 simply measures electrocardiograms and transmits measurement results to the external radio device 200 in the default mode or continuous reproduction mode, it is desirable that the electrocardiogram measurement module 100 can analyze measured electrocardiogram data exceptionally only in a case in which an abnormal electrocardiogram signal is generated and thus electrocardiogram data is intermittently transmitted to the radio device 200 in the device storage mode.

Those skilled in the art will appreciate that the present invention may be carried out in other specific ways than those set forth herein without departing from the spirit and essential characteristics of the present invention. The above embodiments are therefore to be construed in all aspects as illustrative and not restrictive. The scope of the invention should be determined by the appended claims and their legal equivalents, not by the above description, and all changes coming within the meaning and equivalency range of the appended claims are intended to be embraced therein.

While the preferred embodiment(s) of the present disclosure have been described above with reference to the accompanying drawings, the present disclosure is not limited to the above examples. It should be understood by those skilled in the art that various modifications, combinations, sub-combinations and alterations may occur depending on design requirements and other factors within the scope of the appended claims or the equivalents thereof.

1. A wearable integrated electrocardiogram measurement device comprising:
a single patch type electrode sheet having a plurality of electrodes formed on a sheet to be attached to a chest; and
a micro-electrocardiogram measurement module directly attached to and integrated with the electrode sheet and configured to receive electrical signals from the electrodes, to process the received signals and to transmit the processed signals to the outside.

2. The wearable integrated electrocardiogram measurement device according to claim 1, wherein the micro-electrocardiogram measurement module is detachably attached to the electrode sheet through pogo pins.

3. The wearable integrated electrocardiogram measurement device according to claim 1, wherein the electrode sheets include a plurality of electrically connected attachment electrodes and 4-limb electrodes arranged on an integrated sheet.

4. The wearable integrated electrocardiogram measurement device according to claim 3, wherein the attachment electrodes include a first electrode V1 and a second electrode V2 attached to the solar plexus of a patient,
wherein the micro-electrocardiogram measurement module is electrically connected to a center point between the first electrode V1 and the second electrode V2 through a pogo pin contact part electrically connected to the pogo pins.

5. The wearable integrated electrocardiogram measurement device according to claim 4, wherein the micro-electrocardiogram measurement module is connected through the pogo pin contact part using a mounted type coupling method or a clip type coupling method.

6. The wearable integrated electrocardiogram measurement device according to claim 3, wherein the 4-limb electrodes include a first 4-limb electrode RA disposed at a right upper point of a right arm, a second 4-limb electrode RL disposed at a right lower point of a right leg, a third 4-limb electrode LA disposed at a left upper point of a left arm and a fourth 4-limb electrode LL disposed at a left lower point of a left leg.

7. The wearable integrated electrocardiogram measurement device according to claim 6, wherein one or more of the first to fourth 4-limb electrodes are connected to the attachment electrodes through cables.

8. The wearable integrated electrocardiogram measurement device according to claim 1, wherein the micro-electrocardiogram measurement module comprises:
a power management unit configured to provide power to electric devices and to manage a charging state of a battery;
an electrocardiogram sensing processor configured to measure electrocardiogram signals through electrical connection with the electrode sheets and to process measured electrocardiogram signals;
an electrocardiogram storage unit configured to store electrocardiogram data sensed and signal-processed in the electrocardiogram sensing processor;
a display configured to display a state of the electrocardiogram measurement device on a screen;
a wireless communication unit configured to provide an interface for realizing wireless communication with an external terminal; and
a controller configured to control the power management unit, the electrocardiogram sensing processor, the electrocardiogram storage unit, the display and the wireless communication unit.

9. The wearable integrated electrocardiogram measurement device according to claim 8, wherein the electrocardiogram sensing processor acquires 12-channel information through voltages respectively measured by the plurality of electrodes of the electrode sheet.

10. The wearable integrated electrocardiogram measurement device according to claim 8, wherein the display displays power and battery states, electrodes and device attachment states, cardiovascular states of a measured person, a system operation mode or a communication connection state.

11. A wearable wireless 12-channel electrocardiogram system comprising:
a wearable integrated electrocardiogram measurement device including a single electrode sheet having 10 electrodes and capable of being attached to a chest, and a micro-electrocardiogram measurement module detachably attached to and integrated with the electrode sheet, and configured to receive electrical signals from the 10 electrodes, to process the received signals and to transmit the processed signals to the outside; and
a radio device including a controller configured to analyze and process electrocardiogram measurement information received from the wearable integrated electrocardiogram measurement device into 12 channels and to transmit the analyzed and processed electrocardiogram measurement information to an external server.

12. The wearable wireless 12-channel electrocardiogram system according to claim 11, wherein the radio device is implemented through a dedicated application after user authentication.

13. The wearable wireless 12-channel electrocardiogram system according to claim 11, wherein the radio device outputs 12-channel electrocardiogram signals, user cardiovascular state information, status summary, user and device information or time and connection information through a screen.

14. The wearable wireless 12-channel electrocardiogram system according to claim 13, wherein, when the 12 channels include a channel through which no signal is output, the radio device detects the channel and generates an alarm indicating an abnormal electrode attachment state.

15. The wearable wireless 12-channel electrocardiogram system according to claim 13, wherein, when the radio device determines that a cardiovascular state of a user is abnormal, the radio device outputs an alarm indicating the abnormal cardiovascular state and transmits the alarm signal to the outside.

16. The wearable wireless 12-channel electrocardiogram system according to claim 15, wherein the abnormal cardiovascular state corresponds to a case in which a current heart rate or a heart rate variation deviates from a predetermined normal heart rate or heart rate variation.

17. The wearable wireless 12-channel electrocardiogram system according to claim 14, wherein the alarm uses a visual, acoustic or tactile alarm means.

18. The wearable wireless 12-channel electrocardiogram system according to claim 11, wherein the radio device remotely controls setting of an operation mode of the micro-electrocardiogram measurement module and a gain of the micro-electrocardiogram measurement module.

19. The wearable wireless 12-channel electrocardiogram system according to claim 11, wherein the radio device analyzes electrocardiogram measurement information received from the wearable integrated electrocardiogram measurement device through 12 channels to extract a cardiovascular state index.

20. The wearable wireless 12-channel electrocardiogram system according to claim 19, wherein the cardiovascular state index includes a current heart rate, an average heart rate, a maximum heart rate, a minimum heart rate or an instantaneous heart rate.

21. A wearable wireless 12-channel electrocardiogram system comprising:
a wearable integrated electrocardiogram measurement device including a single electrode sheet having 10 electrodes and capable of being attached to a chest, and a micro-electrocardiogram measurement module detachably attached to and integrated with the electrode sheet, and configured to receive electrical signals from the 10 electrodes, to process the received signals and to transmit the processed signals to the outside;
a radio device including a controller configured to analyze and process electrocardiogram measurement information received from the wearable integrated electrocardiogram measurement device into 12 channels and to transmit the analyzed and processed electrocardiogram measurement information to an external server; and
a server configured to receive electrocardiogram signals and cardiovascular state data from the radio device, to store the electrocardiogram signals and cardiovascular state data and to transmit diagnosis results based on the electrocardiogram signals and cardiovascular state data to the radio device,
wherein one of a default mode, a continuous reproduction mode and a device storage mode is realized depending on whether electrocardiogram data is transmitted in real time among the electrocardiogram measurement module, the radio device and the server.

22. The wearable wireless 12-channel electrocardiogram system according to claim 21, wherein the default mode is realized when electrocardiogram data is transmitted in real time between the electrocardiogram measurement module and the radio device, and electrocardiogram data is transmitted in real time between the radio device and the server and stored in the radio device and the server in real time.

23. The wearable wireless 12-channel electrocardiogram system according to claim 21, wherein the continuous reproduction mode is realized when electrocardiogram data is transmitted in real time between the electrocardiogram measurement module and the radio device and stored in the electrocardiogram measurement module and the radio device in real time, and electrocardiogram data or diagnosis results are intermittently transmitted between the radio device and the server and intermittently stored in the radio device and the server.

24. The wearable wireless 12-channel electrocardiogram system according to claim 21, wherein the device storage mode is realized when electrocardiogram data is intermittently transmitted between the electrocardiogram measurement module and the radio device, and electrocardiogram data or diagnosis results are intermittently transmitted between the radio device and the server and intermittently stored in the radio device and the server.

25. The wearable wireless 12-channel electrocardiogram system according to claim 23, wherein the intermittent transmission includes periodic transmission, aperiodic transmission at a desired time or transmission when measured electrocardiogram data is abnormal.
